SEQUENCE LISTING (28 sequences)

SEQ ID NO: 1 - DNA, 738 bp, artificial sequence
  Claudin18.2 scFv1
caggtccaactgcagcagcctggggctgagctggtgaggcctggggcttcagtgaagctgtcctgcaaggcttctggctacaccttcaccagctactggataaactgggtgaagcagaggcctggacaaggccttgagtggatcggaaatatttatccttctgatagttatactaactacaatcaaaagttcaaggacaaggccacattgactgtagacaaatcctccagcacagcctacatgcagctcagcagcccgacatctgaggactctgcggtctattactgtacaagatcgtggaggggtaactcctttgactactggggccaaggcaccactctcacagtctcctcaggtggaggcggttcaggcggaggtggctctggcggtggcggatcggacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccattcacgttcggctcggggacaaagttggaaataaaa

SEQ ID NO: 2 - protein, 246 aa, artificial sequence
  Claudin18.2 scFv1
QVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 3 - DNA, 726 bp, artificial sequence
  CD19 scFv
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 4 - protein, 242 aa, artificial sequence
  CD19 scFv
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 5 - DNA, 747 bp, artificial sequence
  CD22 scFv
caggtgcagctgcagcagtctggacccggcctcgtgaagcctagccagaccctgtctctgacctgcgccatcagcggcgatagcgtgtccagcaatagcgccgcctggaactggatccggcagagcccttctagaggcctggaatggctgggccggacctactaccggtccaagtggtacaacgactacgccgtgtccgtgaagtcccggatcaccatcaaccccgacaccagcaagaaccagttctccctgcagctgaacagcgtgacccccgaggataccgccgtgtactactgcgccagagaagtgaccggcgacctggaagatgccttcgacatctggggccagggcacaatggtcaccgtgtctagcggcagtggaaagggctcaacgtcacccggttccggggaggggtcaactaagggcgatattcagatgacacagagcccctccagcctgtccgcctctgtgggagacagagtgacaatcacctgtcgggcctcccagaccatctggtcctatctgaattggtatcagcagcggcctggcaaggcccccaacctgctgatctatgccgccagctctctgcagtccggcgtgccatctagattcagcggcagaggcagcggcaccgatttcaccctgacaattagcagtctgcaggccgaggacttcgccacctactattgccagcagagctacagcatcccccagaccttcggccagggaacaaaactggaaatcaaa

SEQ ID NO: 6 - protein, 249 aa, artificial sequence
  CD22 scFv
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGSGKGSTSPGSGEGSTKGDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 7 - DNA, 366 bp, artificial sequence
  Claudin18.2 sdAb
caggtccaacttgttgaatcaggcggtggccttgtccagccggggggttccctcaggttgagttgcgccgcttccggttcaattttctcaataaacgctatgggttggtataggcaggcgccggggaagggactggagctcgtagcagcaattaccttcggcggagggtctacaaattatgccgactcagtgaagggaagatttacgattagcagagacaattcaaagaatactctctacttgcaaatgaatagccttagagccgaggacactgctgtttactattgtaatgctgacttgctggttggtggctttcctcgacgaaacgtatattggggccagggcaccctcgtaacggtctccagc

SEQ ID NO: 8 - protein, 122 aa, artificial sequence
  Claudin18.2 sdAb
QVQLVESGGGLVQPGGSLRLSCAASGSIFSINAMGWYRQAPGKGLELVAAITFGGGSTNYADSVKGRFTISRDNSKNTLYLQMNSLRAEDTAVYYCNADLLVGGFPRRNVYWGQGTLVTVSS

SEQ ID NO: 9 - DNA, 639 bp, artificial sequence
  Fc
ctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 232 aa, artificial sequence
  Fc
EPKSQDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 75 bp, artificial sequence
  CD8 transmembrane domain
atctacatctgggcgcccttggccgggacttgtggggtccttctcctgtcactggttatcaccctttactgcaaa

SEQ ID NO: 12 - protein, 25 aa, artificial sequence
  CD8 transmembrane domain
IYIWAPLAGTCGVLLLSLVITLYCK

SEQ ID NO: 13 - DNA, 120 bp, artificial sequence
  4-1BB costimulatory domain
cggggcagaaagaaactcctgtatatattcaaacaaccatttatgagaccagtacaaactactcaagaggaagatggctgtagctgccgatttccagaagaagaagaaggaggatgtgaa

SEQ ID NO: 14 - protein, 40 aa, artificial sequence
  4-1BB costimulatory domain
RGRKKLLYIFKQPFMRPVQTTQEEDGCSCRFPEEEEGGCE

SEQ ID NO: 15 - DNA, 339 bp, artificial sequence
  CD3 signaling domain
ctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 16 - protein, 113 aa, artificial sequence
  CD3 signaling domain
LRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 57 bp, artificial sequence
  IgG linker
gagcccaaatctcaggacaaaactcacacatgcccaccgtgcccagcacctgaactc

SEQ ID NO: 18 - protein, 19 aa, artificial sequence
  IgG linker
EPKSQDKTHTCPPCPAPEL

SEQ ID NO: 19 - DNA, 60 bp, artificial sequence
  CD8 signal peptide
atggccttaccagtgaccgccttgctcctgccgctggccttgctgctccacgccgccagg

SEQ ID NO: 20 - protein, 21 aa, artificial sequence
  CD8 signal peptide
MALPVTALLLPLALLLHAARP

SEQ ID NO: 21 - DNA, 66 bp, artificial sequence
  GM-CSFR signal peptide
atgctgctgctcgtgacaagcctgctgctgtgcgagctgccccaccctgcctttctgctgatcccc

SEQ ID NO: 22 - protein, 22 aa, artificial sequence
  GM-CSFR signal peptide
MLLLVTSLLLCELPHPAFLLIP

SEQ ID NO: 23 - DNA, 66 bp, artificial sequence
  F2A
gtgaaacagactttgaattttgaccttctcaagttggcgggagacgtggagtccaacccagggccg

SEQ ID NO: 24 - protein, 22 aa, artificial sequence
  F2A
VKQTLNFDLLKLAGDVESNPGP

SEQ ID NO: 25 - DNA, 135 bp, artificial sequence
  CD8 hinge region
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 26 - protein, 45 aa, artificial sequence
  CD8 hinge region
TTTPAPRPPTPAPTIASQPLSLRPEACRPAAGGAVHTRGLDFACD

SEQ ID NO: 27 - DNA, 738 bp, artificial sequence
  Claudin18.2 scFv2
cagatccagttggtgcagtctggacctgagctgaagaagcctggagagacagtcaagatctcctgcaaggcttctgggtataccttcacaaactatggaatgaactgggtgaagcaggctccaggaaagggtttaaagtggatgggctggataaacaccaacactggagagccaacctatgctgaagagttcaagggacggtttgccttctctttggaaacctctgccagcactgcctatttgcagatcaacaacctcaaaaatgaggacacggctacatatttctgtgcaagactgggttttggtaatgctatggactactggggtcaaggaacctcagtcaccgtctcctcaggtggaggcggttcaggcggaggtggctctggcggtggcggatcggacattgtgatgacacagtctccatcctccctgactgtgacagcaggagagaaggtcactatgagctgcaagtccagtcagagtctgttaaacagtggaaatcaaaagaactacttgacctggtaccagcagaaaccagggcagcctcctaaactgttgatctactgggcatccactagggaatctggggtccctgatcgcttcacaggcagtggatctggaacagatttcactctcaccatcagcagtgtgcaggctgaagacctggcagtttattactgtcagaatgattatagttatccgctcacgttcggtgctgggaccaagctggagctgaaa

SEQ ID NO: 28 - protein, 246 aa, artificial sequence
  Claudin18.2 scFv2
QIQLVQSGPELKKPGETVKISCKASGYTFTNYGMNWVKQAPGKGLKWMGWINTNTGEPTYAEEFKGRFAFSLETSASTAYLQINNLKNEDTATYFCARLGFGNAMDYWGQGTSVTVSSGGGGSGGGGSGGGGSDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLELK

(19) United States
(12) Patent Application Publication
(10) Pub. No.: US 2023/0242877 A1
(43) Pub. Date: Aug. 3, 2023

(54) IMMUNE CELL COMPRISING CHIMERIC ANTIGEN RECEPTOR AND USE THEREOF

(71) Applicant: NANJING BIOHENG BIOTECH CO., LTD, Nanjing, Jiangsu (CN)

(72) Inventors: Qiping SHI, Nanjing, Jiangsu (CN); Wen JIANG, Nanjing, Jiangsu (CN); Xiaohong HE, Nanjing, Jiangsu (CN); Jiangtao REN, Nanjing, Jiangsu (CN); Yanbin WANG, Nanjing, Jiangsu (CN); Lu HAN, Nanjing, Jiangsu (CN)

(21) Appl. No.: 17/794,031

(22) PCT Filed: Jan. 21, 2021
(86) PCT No.: PCT/CN2021/072978

§ 371 Date: Jul. 20, 2022

(30) Foreign Application Priority Data
Jan. 21, 2020 (CN) 202010072642.8

(51) Int. Cl. C12N 5/0783; C12N 15/63; C07K 16/18; C07K 14/725

(52) U.S. Cl. CPC C12N 5/0636; C12N 15/63; C07K 16/18; C07K 14/7051; C07K 2317/524; C07K 2317/526; C07K 2317/569; C07K 2317/24; C07K 2317/622; C07K 2319/30

(57) ABSTRACT

The invention relates to an engineered immune cell comprising: (a) a first nucleic acid sequence encoding a chimeric antigen receptor or a chimeric antigen receptor encoded thereby, said chimeric antigen receptor comprises a first antigen binding region, a transmembrane domain, and an intracellular signaling domain; and (b) a second nucleic acid sequence encoding an Fc fusion polypeptide or an Fc fusion polypeptide encoded thereby, said Fc fusion polypeptide comprises a second antigen binding region and an Fc region, wherein the first antigen binding region and the second antigen binding region are not scFv at the same time. The invention also relates to compositions comprising the engineered immune cells of the invention, and the use of the engineered immune cells/compositions in the treatment of cancer.

### SEQUENCE LISTING

This application contains a Sequence Listing which has been submitted in ASCII format via EFS-WEB and is hereby incorporated by reference in its entirety. The ASCII copy, dated Dec. 1, 2022, is named “105147-036-Sequence_listing_Dec_1_2022.txt” and is 24 KB.

### FIELD OF THE INVENTION

The invention relates to the field of immunotherapy, particularly relates to immune cells comprising a chimeric antigen receptor (CAR) and uses thereof, especially in the treatment of cancer.

### BACKGROUND OF THE INVENTION

In recent years, cancer immunotherapy technology has developed rapidly, especially chimeric antigen receptor T cell (CAR-T)-related immunotherapy has achieved excellent clinical effects in the treatment of hematological tumors. CAR-T cell immunotherapy is to genetically modify T cells in vitro to recognize tumor antigen, and then re-infuse them into patient to kill cancer cells after expanding to a certain number, so as to achieve the purpose of treating tumor.

There are many types of cells involved in or related to the immune response in the human body, including T lymphocytes (also known as T cells), B lymphocytes (also known as B cells), natural killer cells (NK cells), macrophages, dendritic cells, mast cells, etc. Among them, T cells are the main components of lymphocytes and have a variety of biological functions, such as directly killing target cells, helping or inhibiting B cells to produce antibodies, responding to specific antigens, and producing cytokines. Cellular immunity is the immune response produced by T cells. There are two main effector forms of cellular immunity. One is specific binding with target cells, which destroys the target cell membrane and directly kill the target cells. The other is releasing lymphatic factor, eventually expanding and enhancing the immune effect. NK cells are few in number, but essential for human innate immunity. The recognition of allogeneic antigens by such immune cells does not require the mediation of antibodies and major histocompatibility complex (MHC), and the immune killing response of NK cells is rapid. This broad and rapid immune killing ability of NK cells makes them an ideal immune cell in tumor immune cell therapy. Macrophages have a variety of functions, not only phagocytosing pathogens, but also presenting antigens after ingesting them. There are also a large number of tumor-associated macrophages (TAMs) in the tumor microenvironment. They have a high degree of interaction with tumor cells, tumor stem cells, epidermal cells, fibroblasts, as well as T cells, B cells, NK cells, etc. Dendritic cells (DCs) are the most powerful professional antigen-presenting cells (APCs) in the body, capable of efficiently ingesting, processing and presenting antigens. NK cells and macrophages have significant tumor infiltration advantages and can efficiently present antigens to T cells. Moreover, NK cells also have the effect of activating DC cells.

Therefore, activating NK cells, macrophages, DC cells during CAR-T immunotherapy will help solve many problems existing in CAR-T cell therapy, such as immunosuppression in tumor microenvironment, tumor heterogeneity, and difficulty in T cell infiltration, and significantly improve the overall therapeutic effect.

### SUMMARY OF THE INVENTION

In the first aspect, the invention provides an engineered immune cell comprising:

(a) a first nucleic acid sequence encoding a chimeric antigen receptor or a chimeric antigen receptor encoded thereby, wherein the chimeric antigen receptor comprises a first antigen binding region, a transmembrane domain, and an intracellular signaling domain; and

(b) a second nucleic acid sequence encoding an Fc fusion polypeptide or an Fc fusion polypeptide encoded thereby, wherein the Fc fusion polypeptide comprises a second antigen binding region and an Fc region,

wherein the first antigen binding region and the second antigen binding region are not scFv at the same time.

In one embodiment, the first antigen binding region and the second antigen binding region bind the same antigen. In another embodiment, the first antigen binding region and the second antigen binding region bind different antigens.

In one embodiment, the first and second antigen binding regions are selected from scFv, sdAb and nanobody. Preferably, the first antigen binding region is an scFv and the second antigen binding region is an sdAb or nanobody, or the first antigen binding region is an sdAb or nanobody and the second antigen binding region is an scFv.

In one embodiment, the first and second antigen binding regions are selected from monoclonal antibodies, polyclonal antibodies, recombinant antibodies, human antibodies, humanized antibodies, murine antibodies and chimeric antibodies.

In one embodiment, the first antigen binding region and the second antigen binding region bind to a target selected from TSHR, CD19, CD123, CD22, BAFF-R, CD30, CD171, CS-1, CLL-1, CD33, EGFRvIII, GD2, GD3, BCMA, GPRC5D, Tn Ag, PSMA, ROR1, FLT3, FAP, TAG72, CD38, CD44v6, CEA, EPCAM, B7H3, KIT, IL-13Ra2, mesothelin, IL-11Ra, PSCA, PRSS21, VEGFR2, LewisY, CD24, PDGFR-β, SSEA-4, CD20, Folate receptor α, ERBB2 (Her2/neu), MUC1, EGFR, NCAM, Claudin18.2, Prostase, PAP, ELF2M, Ephrin B2, IGF-I receptor, CAIX, LMP2, gploo, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD 179a, ALK, polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, Podin, HPV E6, E7, MAGE A1, ETV6-AML, spermatin 17, XAGE1, Tie 2, MAD-CT-1, MAD-CT-2, Fos-related Antigen 1, p53, p53 mutant, prostate specific protein, survivin and telomerase, PCTA-1/Galectin 8, MelanA/MART1, Ras mutant, hTERT, sarcoma translocation breakpoint, ML-IAP, ERG (TMPRSS2 ETS fusion gene), NA17, PAX3, androgen receptor, Cyclin B1, MYCN, RhoC, TRP-2, CYP1B 1, BORIS, SART3, PAX5, OY-TES 1, LCK, AKAP-4, SSX2, RAGE-1. Human telomerase reverse transcriptase, RU1, RU2, intestinal carboxylesterase, mut hsp70-2, CD79a, CD79b, CD72, LAIR1, FCAR, LILRA2, CD300LF, CLEC12A, BST2, EMR2, LY75, GPC3, FCRL5, IGLL1, PD1, PDL1, PDL2, TGF β, APRIL, NKG2D, and any combination thereof. Preferably, the target is selected from CD19, CD20, CD22, BAFF-R, CD33, EGFRvIII, BCMA, GPRC5D, PSMA, ROR1, FAP, ERBB2 (Her2/neu), MUC1, EGFR, CAIX, WT1, NY-ESO-1, CD79a, CD79b, GPC3, Claudin18.2, NKG2D and any combination thereof.

In one embodiment, the transmembrane domain is selected from the transmembrane domains of the following proteins: TCR α chain, TCR β chain, TCR γ chain, TCR δ chain, CD3 ζ subunit, CD3 ε subunit, CD3 γ subunit, CD3 δ subunit, CD45, CD4, CD5, CD8 α, CD9, CD16, CD22, CD33, CD28, CD37, CD64, CD80, CD86, CD134, CD137, CD154 and CD278. Preferably, the transmembrane domain is selected from the transmembrane domains of CD8 α, CD4, CD28 and CD278.

In one embodiment, the chimeric antigen receptor comprises an intracellular signaling domain selected from the signaling domains of FcR γ, FcR β, CD3 γ, CD3 δ, CD3 ε, CD3 ζ, CD22, CD79a, CD79b, and CD66d. Preferably, the intracellular signaling domain comprises CD3 ζ signaling domain.

In one embodiment, the chimeric antigen receptor further comprises one or more costimulatory domains. Preferably, the costimulatory domain is a costimulatory signaling domain selected from TLR1, TLR2, TLR3, TLR4, TLR5, TLR6, TLR7, TLR8, TLR9, TLR10, CARD11, CD2, CD7, CD8, CD18 (LFA-1), CD27, CD28, CD30, CD40, CD54 (ICAM), CD83, CD134 (OX40), CD137 (4-1BB), CD270 (HVEM), CD272 (BTLA), CD276 (B7-H3), CD278 (ICOS), CD357 (GITR), DAP10, LAT, NKG2C, SLP76, PD-1, LIGHT, TRIM and ZAP70. Preferably, the costimulatory domain is a costimulatory signaling domain of CD27, CD28, CD134, CD137 or CD278.

In one embodiment, the Fc region comprises a CH2 domain and a CH3 domain, preferably the CH2 and CH3 domains of IgG1.

In one embodiment, the engineered immune cells of the invention comprise a first nucleic acid sequence encoding a chimeric antigen receptor and a second nucleic acid sequence encoding an Fc fusion polypeptide, the first nucleic acid sequence and the second nucleic acid sequence are located in different vectors. In another embodiment, the first nucleic acid sequence and the second nucleic acid sequence are located in the same vector.

In one embodiment, the vectors of the invention are linear nucleic acid molecules, plasmids, retroviruses, lentiviruses, adenoviruses, vaccinia virus, Rous sarcoma virus (RSV), polyoma virus, adeno-associated virus (AAV), bacteriophage, cosmids or artificial chromosomes.

In one embodiment, the vector of the invention further comprises one or more elements selected from an origin of autonomous replication in a host cell, a selectable marker, a restriction enzyme cleavage site, a promoter, a polyadenylation tail (polyA), 3′UTR, 5′UTR, enhancers, terminators, insulators, operons, selectable markers, reporter genes, targeting sequences and protein purification tags.

In one embodiment, the immune cells of the invention are selected from T cells, macrophages, dendritic cells, monocytes, NK cells or NKT cells. Preferably, the T cells are CD4+/CD8+ double positive T cells, CD4+ helper T cells, CD8+ T cells, tumor infiltrating cells, memory T cells, naive T cells, γ δ-T cells or α β-T cells.

In the second aspect, the invention provides a pharmaceutical composition comprising an immune cell of the invention as defined above and one or more pharmaceutically acceptable excipients.

In the third aspect, the invention provides a method of preparing an engineered immune cell, comprising introducing into said immune cells:

(a) a first nucleic acid sequence encoding a chimeric antigen receptor or a chimeric antigen receptor encoded thereby, wherein the chimeric antigen receptor comprises a first antigen binding region, a transmembrane domain, and an intracellular signaling domain; and

(b) a second nucleic acid sequence encoding an Fc fusion polypeptide or an Fc fusion polypeptide encoded thereby, wherein the Fc fusion polypeptide comprises a second antigen binding region and an Fc region,

wherein the first antigen binding region and the second antigen binding region are not scFv at the same time.

In the fourth aspect, the invention provides a kit, comprising:
a vector comprising a first nucleic acid sequence encoding a chimeric antigen receptor comprising a first antigen binding region, a transmembrane domain and an intracellular signaling domain; and
a vector comprising a second nucleic acid sequence encoding an Fc fusion polypeptide comprising a second antigen binding region and an Fc region,

wherein the first antigen binding region and the second antigen binding region are not scFv at the same time.

In the fifth aspect, the invention provides a method of treating a subject suffering from cancer, comprising administering to the subject an effective amount of an immune cell or a pharmaceutical composition according to the invention.

In one embodiment, the cancer is selected from brain glioma, blastoma, sarcoma, leukemia, basal cell carcinoma, biliary tract cancer, bladder cancer, bone cancer, brain and CNS cancer, breast cancer, peritoneal cancer, Cervical cancer, choriocarcinoma, colon and rectal cancer, connective tissue cancer, cancer of the digestive system, endometrial cancer, esophageal cancer, eye cancer, head and neck cancer, stomach cancer, glioblastoma (GBM), liver cancer, liver cells tumor, intraepithelial tumor, kidney cancer, laryngeal cancer, liver tumor, lung cancer, lymphoma, melanoma, myeloma, neuroblastoma, oral cancer, ovarian cancer, pancreatic cancer, prostate cancer, retinoblastoma, rhabdomyosarcoma, rectal cancer, cancer of the respiratory system, salivary gland cancer, skin cancer, squamous cell carcinoma, gastric cancer, testicular cancer, thyroid cancer, uterine or endometrial cancer, malignancies of the urinary system, vulvar cancer and other cancers and sarcomas, B-cell lymphoma, mantle cell lymphoma, AIDS-related lymphoma, and Waldenstrom macroglobulinemia, chronic lymphocytic leukemia (CLL), acute lymphoblastic leukemia (ALL), B-cell acute lymphoblastic leukemia (B-ALL), T-cell acute lymphoblastic leukemia (T-ALL), B-cell prolymphocytic leukemia, blastic plasmacytoid dendritic cell tumor, Burkitt's lymphoma, diffuse large B-cell lymphoma, follicular Lymphoma, Chronic Myeloid Leukemia (CML), Malignant Lymphoproliferative Disorders, MALT Lymphoma, Hairy Cell Leukemia, Marginal Zone Lymphoma, Multiple Myeloma, Myelodysplasia, Plasmablastic Lymphoma, Preleukemia, Plasma Cytoid dendritic cell tumor, and post-transplant lymphoproliferative disorder (PTLD).

### DETAILED DESCRIPTION

Unless otherwise defined, all scientific and technical terms used herein have the same meaning as commonly understood by one of ordinary skill in the art to which this invention belongs.

### Chimeric Antigen Receptor

As used herein, the term “chimeric antigen receptor” or “CAR” refers to an artificially constructed hybrid polypeptide whose basic structure includes an antigen-binding region (eg. an antigen-binding portion of an antibody), a transmembrane domain and intracellular signaling domain. CAR can exploit the antigen-binding properties of monoclonal antibodies to redirect the specificity and reactivity of T cells and other immune cells to selected targets in an MHC-non-restricted manner. Non-MHC-restricted antigen recognition confers CAR-expressing T cells the ability to recognize antigen independent of antigen processing, thus bypassing the primary mechanism of tumor escape. Furthermore, when expressed within T cells, CAR advantageously does not dimerize with the alpha and beta chains of the endogenous T cell receptor (TCR). Typically, the extracellular binding domain of a CAR consists of a single-chain variable fragment (scFv) derived from fusing the variable heavy and light chain regions of a murine or human or chimeric monoclonal antibody. Alternatively, the scFv that can be used is derived from Fab (rather than from antibody, eg, obtained from a Fab library). In various embodiments, such scFvs are fused to the transmembrane domain and subsequently to the intracellular signaling domain. At present, with the development of technology, four generations of different CAR structures have appeared. The intracellular signaling domains of the first-generation CAR only contains primary signaling domains, such as CD3 ζ, so CAR-bearing cells (such as CAR-T cells) have poor activity and short survival time in vivo. The second-generation CAR introduces co-stimulatory domains, such as CD28 or 4-1BB, to enable sustained cell proliferation and enhanced antitumor activity. The third-generation CAR contains two costimulatory domains (eg CD28+4-1BB), and the fourth-generation CAR adds cytokines or costimulatory ligands to further enhance T cell responses, or suicide genes to make CAR-expressing cells self-destruct when needed.

In one embodiment, the chimeric antigen receptor of the invention comprises a first antigen binding region, a transmembrane domain, and an intracellular signaling domain.

As used herein, the term “antigen binding region” refers to any structure or functional variant thereof that can bind an antigen. The antigen binding region can be an antibody structure including, but not limited to, monoclonal antibodies, polyclonal antibodies, recombinant antibodies, human antibodies, humanized antibodies, chimeric antibodies, and functional fragments thereof. For example, the antigen binding region includes but is not limited to single chain antibody (scFv), single domain antibody (sdAb), nanobody (Nb), antigen binding ligand, recombinant fibronectin domain, anticalin and DARPIN, and is preferably selected from scFv, sdAb and Nanobody. In the invention, the antigen binding region may be monovalent or bivalent, and may be monospecific, bispecific or multispecific. In another embodiment, the antigen binding region may also be a specific binding polypeptide or receptor structure of a specific protein, such as PD1, PDL1, PDL2, TGFβ, APRIL and NKG2D.

The term “single-chain antibody” or “scFv” is an antibody in which the variable region of the heavy chain (VH) and the variable region of the light chain (VL) of an antibody are linked by a linker. The optimal length and/or amino acid composition of the linker can be selected. The length of the linker significantly affects the variable region folding and interaction of scFv. In fact, intrachain folding can be prevented if shorter linkers are used (eg between 5-10 amino acids). For selection of linker size and composition, see, eg. Hollinger et al., 1993 Proc Natl Acad. Sci. U.S.A. 90:6444-6448; US Patent Application Publication No. 2005/0100543, 2005/0175606, 2007/0014794; and PCT Publication No. WO2006/020258 and WO2007/024715, the entire contents of which are incorporated herein by reference.

The term “single domain antibody” or “sdAb” refers to an antibody that is naturally devoid of a light chain, which contains only one variable heavy chain region (VHH) and two conventional CH2 and CH3 regions, also referred to as “Heavy chain antibody”.

The term “Nanobody” or “Nb” refers to a single cloned and expressed VHH structure, which has structural stability and antigen-binding activity comparable to the original heavy chain antibody, and is currently known as the smallest unit capable of binding target antigens.

The term “functional variant” or “functional fragment” refers to a variant comprising substantially the amino acid sequence of the parent but containing at least one amino acid modification (ie, substitution, deletion or insertion) compared to the parent amino acid sequence, provided that the variant retains the biological activity of the parent amino acid sequence. In one embodiment, the amino acid modification is preferably a conservative modification.

As used herein, the term “conservative modification” refers to amino acid modification that does not significantly affect or alter the binding characteristics of an antibody or antibody fragment containing the amino acid sequence. These conservative modifications include amino acid substitutions, additions and deletions. Modifications can be introduced into the chimeric antigen receptor or Fc fusion polypeptides of the invention by standard techniques known in the art, such as site-directed mutagenesis and PCR-mediated mutagenesis. Conservative amino acid substitutions are substitutions in which amino acid residues are replaced by amino acid residues having similar side chains. Families of amino acid residues with similar side chains have been defined in the art, including basic side chains (eg, lysine, arginine, histidine), acidic side chains (eg, aspartic acid, glutamic acid)), uncharged polar side chains (e.g. glycine, asparagine, glutamine, serine, threonine, tyrosine, cysteine), non-polar side chains (e.g. alanine, valine) acid, leucine, isoleucine, proline, phenylalanine, methionine, tryptophan), beta-branched side chains (e.g. threonine, valine, isoleucine) and aromatic side chains (eg tyrosine, phenylalanine, tryptophan, histidine). Conservative modifications can be selected, for example, based on similarity in polarity, charge, solubility, hydrophobicity, hydrophilicity, and/or the amphipathic nature of the residues involved.

Thus, a “functional variant” or “functional fragment” has at least 75%, preferably at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with the parent amino acid sequence, and retain the biological activity, eg, binding activity.

As used herein, the term “sequence identity” refers to the degree to which two (nucleotide or amino acid) sequences have identical residues at the same positions in an alignment, and is usually expressed as percentage. Preferably, identity is determined over the entire length of the sequences being compared. Therefore, two copies with the exact same sequence have 100% identity. Those skilled in the art will recognize that some algorithms can be used to determine sequence identity using standard parameters, such as Blast (Altschul et al. (1997) Nucleic Acids Res. 25:3389-3402), Blast2 (Altschul et al. (1990) J. Mol. Biol. 215: 403-410), Smith-Waterman (Smith et al. (1981) J. Mol. Biol. 147: 195-197) and ClustalW.

In one embodiment, the antigen binding region of the invention binds to one or more targets selected from TSHR, CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, mesothelin, IL-1 1Ra, PSCA, PRSS21, VEGFR2, LewisY, CD24, PDGFR-β, SSEA-4, CD20, Folate receptor α, ERBB2 (Her2/neu), MUC1, EGFR, NCAM, Prostase, PAP, ELF2M, Ephrin B2, IGF-I receptor, CAIX, LMP2, gplOO, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD 179a, ALK, polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, Bean protein, HPV E6, E7, MAGE A1, ETV6-AML, sperm protein 17, XAGE1, Tie 2, MAD-CT-1, MAD-CT-2, Fos-associated antigen 1, p53, p53 mutants, prostate specific protein, survivin and telomerase, PCTA-1/Galectin 8, MelanA/MART1, Ras mutant, hTERT, sarcoma translocation breakpoint, ML-IAP, ERG (TMPRSS2 ETS fusion gene), NA17, PAX3, Androgen Receptor, Cyclin B1, MYCN, RhoC, TRP-2, CYP1B 1, BORIS, SART3, PAX5, OY-TES 1, LCK, AKAP-4, SSX2, RAGE-1, Human Telomerase Reverse Transcription Enzymes, RU1, RU2, intestinal carboxylesterase, mut hsp70-2, CD79a, CD79b, CD72, LAIR1, FCAR, LILRA2, CD300LF, CLEC12A, BST2, EMR2, LY75, GPC3, FCRL5, IGLL1, PD1, PDL1, PDL2, TGF β, APRIL, NKG2D and any combination of them. Preferably, the target is selected from: CD19, CD20, CD22, BAFF-R, CD33, EGFRvIII, BCMA, GPRC5D, PSMA, ROR1, FAP, ERBB2 (Her2/neu), MUC1, EGFR, CAIX, WT1, NY-ESO-1, CD79a, CD79b, GPC3, Claudin18.2, NKG2D and any combination thereof.

As used herein, the term “transmembrane domain” refers to a polypeptide structure which is capable of expressing chimeric antigen receptors on the surface of immune cells (eg, lymphocytes, NK cells, or NKT cells) and directing immune cells against target cells. The transmembrane domain can be natural or synthetic, and can be derived from any membrane-bound or transmembrane protein. The transmembrane domain is capable of signaling when the chimeric receptor polypeptide binds to the target antigen. Transmembrane domains particularly useful in the invention may be derived from, for example, TCR α chain, TCR β chain, TCR γ chain, TCR δ chain, CD3 ζ subunit, CD3 ε subunit, CD3 γ subunit, CD3 δ subunit, CD45, CD4, CD5, CD8 α, CD9, CD16, CD22, CD33, CD28, CD37, CD64, CD80, CD86, CD134, CD137, CD154 and their functional fragments. Alternatively, the transmembrane domain may be synthetic and may contain predominantly hydrophobic residues such as leucine and valine. Preferably, the transmembrane domain is derived from a human CD8 alpha chain, which has at least 70%, preferably at least 80%, more preferably at least 90%, 95%, 97% or 99% or 100% sequence identity with the amino acid sequence shown in SEQ ID NO: 12 or the nucleotide sequence shown in SEQ ID NO: 11.

In one embodiment, the chimeric antigen receptor of the invention may further comprise a hinge region between the first antigen binding region and the transmembrane domain. As used herein, the term “hinge region” generally refers to any oligopeptide or polypeptide that functions to link the transmembrane domain to the antigen binding region. Specifically, the hinge region serves to provide greater flexibility and accessibility to the antigen binding region. The hinge region may comprise up to 300 amino acids, preferably 10 to 100 amino acids and most preferably 25 to 50 amino acids. The hinge region may be derived from all or part of a native molecule, such as from all or part of the extracellular region of CD8, CD4 or CD28, or from all or part of an antibody constant region. Alternatively, the hinge region may be a synthetic sequence corresponding to a naturally occurring hinge sequence, or may be a fully synthetic hinge sequence. In a preferred embodiment, the hinge region comprises a hinge region portion of a CD8 α chain, Fc γ RIII α receptor, IgG4 or IgG1, more preferably a hinge of CD8 α, and has at least 70%, preferably at least 80%, more preferably at least 90%, 95%, 97% or 99% or 100% sequence identity with the amino acid sequence shown in SEQ ID NO:26 or the nucleotide sequence shown in SEQ ID NO: 25.

As used herein, the term “intracellular signaling domain” refers to the portion of a protein that transduces effector function signals and directs a cell to perform a specified function. The intracellular signaling domain is responsible for intracellular signaling after antigen binding to the antigen binding region, resulting in the activation of immune cells and immune responses. In other words, the intracellular signaling domain is responsible for activating at least one of the normal effector functions of the immune cells in which the CAR is expressed. For example, the effector function of T cells can be cytolytic activity or helper activity, including secretion of cytokines.

In one embodiment, the chimeric antigen receptors of the invention comprise intracellular signaling domains that may be cytoplasmic sequences of T cell receptors and co-receptors, as well as any derivatives or variants of these sequences and any synthetic sequences with the same or similar function, which act together upon antigen-receptor binding to initiating signaling. Intracellular signaling domains comprise two distinct types of cytoplasmic signal sequences: those that initiate antigen-dependent primary activation, and those that act in an antigen-independent manner to provide secondary or costimulatory signals. Primary cytoplasmic signal sequences can contain a number of immunoreceptor tyrosine-based Activation Motifs (ITAMs). Non-limiting examples of intracellular signaling domains of the invention include, but are not limited to, those derived from FcR γ, FcR β, CD3 γ, CD3 δ, CD3 ε, CD3 ζ, CD22, CD79a, CD79b, and CD66d. In a preferred embodiment, the signaling domain of the CAR of the invention may comprise a CD3 ζ signaling domain which has at least 70%, preferably at least 80%, more preferably at least 90%, 95%, 97% or 99% or 100% sequence identity with the amino acid sequence shown in SEQ ID NO:16 or the nucleotide sequence shown in SEQ ID NO: 15.

In one embodiment, the chimeric antigen receptor of the invention further comprises one or more costimulatory domains. A costimulatory domain may be an intracellular functional signaling domain from a costimulatory molecule, which may comprise the entire intracellular portion of a costimulatory molecule, or a functional fragment thereof. A “costimulatory molecule” refers to a cognate binding partner that specifically binds to a costimulatory ligand on a T cell, thereby mediating a costimulatory response (eg, proliferation) of the T cell. Costimulatory molecules include, but are not limited to, MHC class 1 molecules, BTLA and Toll ligand receptors. Non-limiting examples of costimulatory domains of the invention include, but are not limited to, costimulatory signaling domains derived from TLR1, TLR2, TLR3, TLR4, TLR5, TLR6, TLR7, TLR8, TLR9, TLR10, CARD11, CD2, CD7, CD8, CD18 (LFA-1), CD27, CD28, CD30, CD40, CD54 (ICAM), CD83, CD134 (OX40), CD137 (4-1BB), CD270 (HVEM), CD272 (BTLA), CD276 (B7-H3), CD278 (ICOS), CD357 (GITR), DAP10, LAT, NKG2C, SLP76, PD-1, LIGHT, TRIM and ZAP70. Preferably, that co-stimulatory domain of the CAR of the invention is a 4-1BB and/or CD28 fragment, more preferably has at least 70%, preferably at least 80%, more preferably at least 90%, 95%, 97% or 99% or 100% sequence identity with the amino acid sequence shown in SEQ ID NO:14 or the nucleotide sequence shown in SEQ ID NO: 13.

In a preferred embodiment, the chimeric antigen receptor of the invention comprises a CD8 α transmembrane domain, a 4-1BB costimulatory domain, and a CD3 ζ signaling domain. More preferably, the chimeric antigen receptor further comprises a CD28 costimulatory domain, a CD8 α hinge region, or both.

### Fc Fusion Polypeptide

As used herein, the term “Fc fusion polypeptide” is a recombinant polypeptide comprising an Fc region and a second antigen binding region, the antigen binding region as defined above. When normally expressed and secreted, the Fc fusion polypeptides of the invention can bind to Fc receptors on the surface of other immune cells such as macrophages, NK cells, dendritic cells, etc., so as to recruit these immune cells to additionally kill target cells or play the role of antigen presentation, thus to expand the killing effect of CAR cells. In addition, the Fc fusion polypeptide of the invention can also provide additional antigen binding regions, ie, provide individual target cell killing capabilities, as well as diverse antigen targeting.

In one embodiment, the second antigen-binding region included in the Fc fusion polypeptide and the first antigen-binding region included in the above-mentioned CAR are not scFv at the same time. Because the inventor unexpectedly found that when the two are simultaneously scFv, the Fc fusion polypeptide cannot be secreted normally, thereby affecting the recruitment effect of other immune cells, which may be due to the adhesion effect formed between the two scFv structures.

In one embodiment, the first and second antigen binding regions are selected from scFvs, sdAbs and Nanobodies. More preferably, the first antigen binding region is an scFv and the second antigen binding region is an sdAb or Nanobody, or the first antigen binding region is an sdAb or Nanobody and the second antigen binding region is an scFv.

In one embodiment, the first and second antigen binding regions may bind the same antigen or different antigens. According to a specific embodiment, the first and/or second antigen binding region binds Claudin18.2, CD19 or CD22. According to a more specific embodiment, the first antigen binding region comprises SEQ ID NO:8 and the second antigen binding region comprises SEQ ID NO:2, 4, 6 or 28; alternatively, the first antigen binding region comprises SEQ ID NO:2, 4, 6 or 28, the second antigen binding region comprises SEQ ID NO:8. According to another specific embodiment, the first and/or second antigen binding region comprises a functional variant of the above-mentioned sequence, for example having the same CDRs and has at least 80%, at least 85%, at least 90%, at least 95%, at least 98% or at least 99% sequence identity with SEQ ID NO: 2, 4, 6, 8 or 28. The functional variant may be formed by substitution, addition or deletion of one or more (eg, 1 to 10, 1 to 5, or 1 to 3) amino acid residues. In particular, the functional variant has the same or similar function and activity as SEQ ID NO: 2, 4, 6, 8 or 28.

As used herein, the term “Fc region” refers to the C-terminal region of an immunoglobulin heavy chain, which contains at least part of the constant region. The Fc region has no antigen-binding activity and is the site where immunoglobulins interact with effector molecules or cells. The term includes native Fc regions and variant Fc regions. “Native Fc region” refers to a molecule or sequence comprising a non-antigen-binding fragment, whether in monomeric or multimeric form, produced by digestion of an intact antibody. The immunoglobulin source from which the native Fc region is derived is preferably derived from humans. Native Fc fragments are composed of monomeric polypeptides that can be linked in dimeric or multimeric form by covalent linkages (eg, disulfide bonds) and non-covalent linkages. Depending on the class (eg IgG, IgA, IgE, IgD, IgM) or subtype (eg IgG1, IgG2, IgG3, IgA1, IgGA2), there are 1-4 intermolecular disulfides between the monomeric subunits of native Fc molecules. An example of a native Fc fragment is the disulfide-linked dimer produced by papain digestion of IgG (see Ellison et al. (1982) Nucleic Acids Res. 10:4071-9). The term “native Fc” as used herein generally refers to monomeric, dimeric and multimeric forms. A “variant Fc region” refers to an amino acid sequence that differs from that of a “native” or “wild-type” Fc region due to at least one amino acid modification, also referred to as an “Fc variant”. Thus, “Fc region” also includes single-chain Fc (scFc), ie, a single-chain Fc region consisting of two Fc monomers joined by a polypeptide linker, which is capable of naturally folding into a functional dimeric Fc region. In one embodiment, the variant Fc region has at least about 80%, at least about 85%, at least about 90%, more preferably at least about 95%, 96%, 97%, 98%, or at least about 99% sequence identity with the native Fc region.

In a specific embodiment, the Fc region contained in the Fc fusion polypeptide of the invention is preferably derived from IgG. Human IgG has four subtypes: IgG1, IgG2, IgG3, and IgG4 based the antigenic differences of r-chains in IgG molecules, of which IgG1 has the highest distribution abundance in serum. The constant region sequences of these four isoforms are highly homologous, but each isoform is specific regarding antigen binding, immune complex formation, complement activation, triggering effector cells, half-life, and placental transport properties. In a preferred embodiment, the Fc region contained in the Fc fusion polypeptide of the invention is preferably derived from IgG1, so as to enhance the affinity of the Fc region with the receptor, thereby improving the recruitment efficiency of other immune cells.

In one embodiment, the Fc region of the invention refers to a constant region that does not include CH1. For example, in the case of IgA, IgD and IgG, the Fc region comprises the constant domains CH2 and CH3; in the case of IgE and IgM, the Fc region comprises the constant domains CH2, CH3 and CH4. In addition, for IgG, the Fc region may also comprise the lower hinge region between CH1 and CH2. Therefore, preferably, the Fc region of the invention comprises CH2 and CH3 of IgG1, more preferably also the lower hinge region between CH1 and CH2. In a specific embodiment, the Fc region has the same or similar receptor binding activity as the amino acid sequence shown in SEQ ID NO: 10, and has at least 70%, preferably at least 80%, more preferably at least 90%, 95%, 97% or 99% or 100% sequence identity with the amino acid sequence shown in SEQ ID NO: 10.

### Engineered Immune Cells and Preparation Method Thereof

The invention provides engineered immune cells comprising a chimeric antigen receptor or a nucleic acid encoding thereby, and an Fc fusion polypeptide comprising an Fc region or a nucleic acid encoding thereby, also referred to herein as Fite-CAR (Fc induced Target cell engaging Chimeric Antigen Receptor).

As used herein, the term “immune cell” refers to any cell of the immune system that has one or more effector functions (eg, cytotoxic cell killing activity, secretion of cytokines, induction of ADCC and/or CDC). For example, the immune cells can be T cells, macrophages, dendritic cells, monocytes, NK cells, and/or NKT cells. Preferably, the immune cells are T cells. The T cells can be any T cells, such as T cells cultured in vitro, eg, primary T cells, or T cells from T cell lines cultured in vitro, such as Jurkat, SupT1, etc., or T cells obtained from a subject. Examples of subjects include humans, dogs, cats, mice, rats, and transgenic species thereof. T cells can be obtained from a variety of sources, including peripheral blood mononuclear cells, bone marrow, lymph node tissue, cord blood, thymus tissue, tissue from sites of infection, ascites, pleural effusion, spleen tissue, and tumors. T cells can also be concentrated or purified. T cells can be of any type and at any stage of development, including, but not limited to, CD4+/CD8+ double positive T cells, CD4+ helper T cells (eg, Th1 and Th2 cells), CD8+ T cells (eg, cytotoxic T cells), tumor infiltrating cells, memory T cells, naive T cells, γ δ-T cells, α β-T cells, etc. In a preferred embodiment, the immune cells are human T cells. A variety of techniques known to those skilled in the art can be used, such as Ficoll separation to obtain T cells from blood of subjects. In the invention, immune cells are engineered to express chimeric antigen receptor and Fr fusion polypeptide.

The first nucleic acid sequence encoding a chimeric antigen receptor and the second nucleic acid sequence encoding an Fc fusion polypeptide can be introduced into immune cells by conventional methods known in the art (eg, by transduction, transfection, transformation, etc.) such that the chimeric antigen receptor and Fc fusion polypeptide of the invention are expressed in the cells. “Transfection” is a process of introducing nucleic acid molecules or polynucleotides (including vectors) into target cells. One example is RNA transfection, that is, a process of introducing RNA (such as RNA transcribed in vitro, ivt RNA) into host cells. The term is mainly used for non-viral methods in eukaryotic cells. The term “transduction” is generally used to describe virus-mediated transfer of nucleic acid molecules or polynucleotides. Transfection of animal cells typically involves opening transient pores or “holes” in the cell membrane to allow uptake of materials. Transfection can be performed using calcium phosphate, by electroporation, cell extrusion, or mixing cationic lipids with materials to create liposomes that fuse with cell membranes and deposit their cargo inside. Exemplary techniques for transfecting eukaryotic host cells include lipid vesicle-mediated uptake, heat shock-mediated uptake, calcium phosphate-mediated transfection (calcium phosphate/DNA co-precipitation), microinjection, and electroporation. perforation. The term “transformation” is used to describe the non-viral transfer of nucleic acid molecules or polynucleotides (including vectors) into bacteria, but also into non-animal eukaryotic cells (including plant cells). Thus, transformation is the genetic alteration of a bacterial or non-animal eukaryotic cell, which is produced by the direct uptake of the cell membrane from its surroundings and subsequent incorporation of exogenous genetic material (nucleic acid molecules). Conversion can be achieved by artificial means. For transformation to occur, the cells or bacteria must be in a competent state. For prokaryotic transformation, techniques can include heat shock-mediated uptake, fusion of bacterial protoplasts with intact cells, microinjection, and electroporation. Techniques for plant transformation include Agrobacterium-mediated transfer (such as by A. tumefaciens), electroporation, microinjection, and polyethylene glycol-mediated uptake.

In one embodiment, the first nucleic acid sequence encoding the chimeric antigen receptor and the second nucleic acid sequence encoding the Fc fusion polypeptide are located in the same vector. For example, the chimeric antigen receptor and the Fc fusion polypeptide of the invention can be expressed independently without affecting each other by inserting a nucleic acid encoding 2A peptide between the two nucleic acid sequences. As used herein, the term “2A peptide” is a cis-acting hydrolase element (CHYSE) originally discovered in foot-and-mouth disease virus (FMDV). The average length of 2A peptide is 18-22 amino acids. The 2A peptide can be cleaved from its last two amino acids at c-terminal by ribosomal jump during protein translation. Specifically, the peptide chain binding group between glycine and proline is damaged at the 2A site, which can trigger ribosome jumping and start translation from the second codon, thereby achieving independent expression of two proteins in one transcription unit. This 2A peptide-mediated cleavage is widespread in eukaryotic animal cells. The higher splicing efficiency of 2A peptide and its ability to promote the balanced expression of upstream and downstream genes can improve the expression efficiency of heterologous polyproteins (such as cell surface receptors, cytokines, immunoglobulins, etc.). Common 2A peptides include, but are not limited to, P2A, T2A, E2A, F2A, and the like. In another embodiment, the first nucleic acid sequence encoding the chimeric antigen receptor and the second nucleic acid sequence encoding the Fc fusion polypeptide are located in different vectors.

As used herein, the term “vector” is a nucleic acid molecule that acts as a mediator for the transfer of (exogenous) genetic material into immune cells, where the nucleic acid molecule can be replicated and/or expressed.

Vectors generally include targeting vectors and expression vectors. The term “targeting vector” is a medium that delivers an isolated nucleic acid to the interior of a cell by, for example, homologous recombination or a hybrid recombinase using a specific targeting sequence at the site. The term “expression vector” is a vector for transcription of heterologous nucleic acid sequences, such as those encoding the chimeric antigen receptor and Fc fusion polypeptides of the invention, in suitable immune cells and translation of their mRNA. Suitable carriers for use in the invention are known in the art and many are commercially available. In one embodiment, vectors of the invention include, but are not limited to, linear nucleic acid molecules (eg, DNA or RNA), plasmids, viruses (eg, retroviruses, lentiviruses, adenoviruses, vaccinia virus, Rous sarcoma virus (RSV, multiple Oncoviruses and Adeno-Associated Viruses, etc.), phages, phagemids, cosmids, and artificial chromosomes (including BAC and YAC). The vector itself is usually a nucleotide sequence, usually a DNA sequence containing an insert (transgene) and larger sequences that serve as the “backbone” of the vector. Engineered vectors also typically contain an origin for autonomous replication in immune cells (if stable expression of the polynucleotide is needed), a selectable marker, and restriction enzyme cleavage sites (such as multiple cloning sites, MSC). The vector may additionally comprise a promoter, polyadenylation tail (polyA), 3′ UTR, enhancer, terminator, insulator, operon, selectable marker, reporter gene, targeting sequence and/or protein purification tags, etc. In a specific embodiment, said vector is an in vitro transcribed vector. In one embodiment, the immune cells of the invention further comprise at least one inactivated gene selected from: CD52, GR, TCR α, TCR β, CD3 γ, CD3 δ, CD3 ε, CD247 ζ, HLA-I, HLA-II genes, immune checkpoint genes such as PD1 and CTLA-4. More particularly, at least the TCR α or TCR β gene in the immune cells is inactivated. This inactivation renders the TCR non-functional in the cell. This strategy is particularly useful for avoiding graft-versus-host disease (GvHD). Methods of inactivating a gene are known in the art, eg, by meganucleases, zinc finger nucleases, TALE nucleases, or Cas enzyme mediated DNA fragmentation in the CRISPR system, thereby disrupting the expression of the gene.

### Pharmaceutical Compositions and Kits

The invention also provides a pharmaceutical composition comprising the engineered immune cells of the invention as an active agent, with one or more pharmaceutically acceptable excipients. Therefore, the invention also encompasses the use of the engineered immune cells in the preparation of pharmaceutical compositions or medicaments.

As used herein, the term “pharmaceutically acceptable excipient” means one that is pharmacologically and/or physiologically compatible with the subject and the active ingredient (ie, capable of eliciting the desired therapeutic effect without carriers and/or excipients that cause any undesired local or systemic effects), which are well known in the art (see, eg, Remington's Pharmaceutical Sciences. Edited by Gennaro A R, 19th ed. Pennsylvania: Mack Publishing Company, 1995). Examples of pharmaceutically acceptable excipients include, but are not limited to, fillers, binders, disintegrants, coatings, adsorbents, antiadherents, glidants, antioxidants, flavoring agents, colorants, Sweeteners, solvents, co-solvents, buffers, chelating agents, surfactants, diluents, wetting agents, preservatives, emulsifiers, coating agents, isotonic agents, absorption delaying agents, stabilizers and tonicity modifiers. It is known to those skilled in the art to select suitable excipients to prepare the desired pharmaceutical compositions of the invention. Exemplary excipients for use in the pharmaceutical compositions of the invention include saline, buffered saline, dextrose and water. In general, the selection of suitable excipients depends, among other things, on the active agent used, the disease to be treated and the desired dosage form of the pharmaceutical composition.

The pharmaceutical composition according to the invention may be suitable for various routes of administration. Typically, administration is accomplished parenterally. Parenteral delivery methods include topical, intraarterial, intramuscular, subcutaneous, intramedullary, intrathecal, intraventricular, intravenous, intraperitoneal, intrauterine, intravaginal, sublingual or intranasal administration.

The pharmaceutical compositions of the invention can also be prepared in various forms, such as solid, liquid, gaseous or lyophilized forms, in particular ointments, creams, transdermal patches, gels, powders, tablets, solutions, aerosols, granules, pills, suspensions, emulsions, capsules, syrups, elixirs, extracts, tinctures or liquid extract extracts, or those particularly suitable for the desired method of administration. Processes known in the invention for the manufacture of pharmaceuticals may include, for example, conventional mixing, dissolving, granulating, dragee-making, milling, emulsifying, encapsulating, entrapping, or lyophilizing. Pharmaceutical compositions comprising immune cells such as those described herein are typically provided in solution and preferably comprise a pharmaceutically acceptable buffer.

The pharmaceutical composition of the invention may also be administered in combination with one or more other agents suitable for the treatment and/or prevention of the disease to be treated. Preferred examples of agents suitable for combination include known anticancer drugs such as cisplatin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetate glucuronate, auristatin E, vincristine and doxorubicin; peptide cytotoxins such as ricin, diphtheria toxin, Pseudomonas bacterial exotoxin A, DNase and RNase; radionuclides such as iodine 131, rhenium 186, indium 111, iridium 90, bismuth 210 and 213, actinium 225 and astatine 213; prodrugs, such as antibody-directed enzyme prodrugs; immunostimulants, such as IL-2, chemokines such as IL-8, Platelet factor 4, melanoma growth-stimulating protein, etc.; antibodies or fragments thereof, such as anti-CD3 antibodies or fragments thereof, complement activators, heterologous protein domains, homologous protein domains, viral/bacterial protein domains and viral/bacterial peptides.

The invention also provides a kit comprising one or more vectors, wherein the vector comprises: (a) a first nucleic acid sequence encoding a chimeric antigen receptor, the chimeric antigen receptor comprising a first an antigen binding region, a transmembrane domain, and an intracellular signaling domain; and (b) a second nucleic acid sequence encoding an Fc fusion polypeptide, the Fc fusion polypeptide comprising a second antigen binding region and an Fc region, wherein the first An antigen binding region and a second antigen binding region are not scFvs at the same time. “Vector” is defined as above.

### Method for Preparing Engineered Immune Cells

The invention also provides a method for preparing engineered immune cells, comprising introducing the chimeric antigen receptor and Fc fusion of the invention or their coding nucleic acid sequences into immune cells, such that the immune cells express chimeric antigen receptors and Fc fusion polypeptides of the invention.

In one embodiment, the immune cells are human immune cells, more preferably human T cells, macrophages, dendritic cells, monocytes, NK cells and/or NKT cells.

Methods for introducing nucleic acids or vectors into immune cells and expressing them are known in the art. For example, nucleic acids or vectors can be introduced into immune cells by physical methods such as calcium phosphate precipitation, lipofection, particle bombardment, microinjection, electroporation, and the like. Alternatively, chemical methods can also be choosed, such as by colloidal dispersion systems, such as macromolecular complexes, nanocapsules, microspheres, beads, and lipid-based systems, including oil-in-water emulsions, micelles, mixed micelles, and lipids body into a nucleic acid or vector. In addition, nucleic acids or vectors can also be introduced by biological methods. For example, viral vectors, especially retroviral vectors and the like, have become the most common method for inserting genes into mammalian, eg, human cells. Other viral vectors can be derived from lentivirus, poxvirus, herpes simplex virus I, adenovirus, adeno-associated virus, and the like.

After the nucleic acid or vector is introduced into immune cells, those skilled in the art can expand and activate the immune cells by conventional techniques.

### Therapeutic Application

The invention also provides a method of treating a subject suffering from cancer, comprising administering to the subject an effective amount of the immune cells or the pharmaceutical composition of the invention.

In one embodiment, an effective amount of an immune cell and/or pharmaceutical composition of the invention is administered directly to a subject. In another embodiment, the method of treatment of the invention is an ex vivo treatment. Specifically, the method comprises the steps of: (a) providing a sample of the subject comprising immune cells; (b) introducing the chimeric antigen receptor and Fc fusion polypeptide of the invention into the immune cells in vitro to obtain modified immune cells, (c) administering the modified immune cells to a subject in need thereof. Preferably, the immune cells provided in step (a) are selected from T cells, NK cells and/or NKT cells; and the immune cells can be obtained from a subject's sample (especially a blood sample) by conventional methods known in the art. However, other immune cells capable of expressing the chimeric antigen receptor and Fc fusion polypeptides of the invention and performing the desired biological effector functions as described herein may also be used. Furthermore, the immune cells are typically selected to be compatible with the subject's immune system, ie preferably the immune cells do not elicit an immunogenic response. For example, “universal recipient cells,” ie, universally compatible lymphocytes that can be grown and expanded in vitro and can perform the desired biological effector function. The use of such cells would not require obtaining and/or providing the subject's own lymphocytes. The ex vivo introduction of step (c) can be carried out by introducing a nucleic acid or vector as described herein into immune cells via electroporation or by infecting immune cells with a viral vector such as a lentiviral vector, adenovirus viral vector, adeno-associated viral vector or retroviral vector. Other conceivable methods include the use of transfection reagents (such as liposomes) or transient RNA transfection.

In one embodiment, the immune cells are autologous or allogeneic cells, preferably T cells, macrophages, dendritic cells, monocytes, NK cells and/or NKT cells, more preferably T cells, NK cells or NKT cells.

As used herein, the term “autologous” refers to any material derived from an individual that will later be re-introduced into that same individual.

As used herein, the term “allogeneic” refers to any material derived from a different animal or different patient of the same species as the individual into which the material is introduced. Two or more individuals are considered allogeneic to each other when the genes at one or more loci are different. In some cases, allogeneic material from individuals of the same species may be genetically different enough for antigenic interactions to occur.

As used herein, the term “subject” is a mammal, which can be a human, a non-human primate, a mouse, a rat, a dog, a cat, a horse, or a cow, but is not limited to these examples. Mammals other than human can advantageously be used as subjects representing animal models of cancer. Preferably, the subject is a human.

In one embodiment, the disease is cancer associated with expression of the target bind to the antigen binding region. For example, such cancers include, but are not limited to: brain glioma, blastoma, sarcoma, leukemia, basal cell carcinoma, biliary tract cancer, bladder cancer, bone cancer, brain and CNS cancer, breast cancer, peritoneal cancer, cervical cancer, choriocarcinoma, colon and rectal cancer, connective tissue cancer, cancer of the digestive system, endometrial cancer, esophageal cancer, eye cancer, head and neck cancer, stomach cancer (including gastrointestinal cancer), glioblastoma (GBM), Liver cancer, hepatocellular tumor, intraepithelial tumor, kidney cancer, laryngeal cancer, liver tumor, lung cancer (such as small cell lung cancer, non-small cell lung cancer, adenocarcinoma and squamous lung cancer), lymphoma (including Hodgkin lymphoma and non-Hodgkin lymphoma), melanoma, myeloma, neuroblastoma, oral cancer (e.g., lips, tongue, mouth, and pharynx), ovarian cancer, pancreatic cancer, prostate cancer, retinoblastoma, rhabdomyosarcoma, rectum cancer, cancer of the respiratory system, salivary gland cancer, skin cancer, squamous cell cancer, stomach cancer, testicular cancer, thyroid cancer, uterine or endometrial cancer, malignant tumors of the urinary system, vulvar cancer and other cancers and sarcomas, and B cells Lymphomas (including low-grade/follicular non-Hodgkin lymphoma (NHL), small lymphocytic (SL) NHL, intermediate-grade/follicular NHL, intermediate-grade diffuse NHL, high-grade immunoblastic NHL, high-grade Lymphoblastic NHL, high-grade small non-cleaving cell NHL, bulky NHL), mantle cell lymphoma, AIDS-related lymphoma, and Waldenstrom macroglobulinemia, chronic lymphocytic leukemia (CLL), acute lymphoblastic leukemia (ALL), B-cell acute lymphoblastic leukemia (B-ALL), T-cell acute lymphoblastic leukemia (T-ALL), B-cell prolymphocytic leukemia, blastic plasmacytoid dendritic cell tumor, Burkitt lymphoma, diffuse large B-cell lymphoma, follicular lymphoma, chronic myeloid leukemia (CML), malignant lymphoproliferative disorders, MALT lymphoma, hairy cell leukemia, marginal zone lymphoma, multiple myeloma, myelodysplasia, plasmablastic lymphoma, preleukemia, plasmacytoid dendritic cell tumor, and post-transplant lymphoproliferative disorder (PTLD); and other diseases associated with target expression. Preferably, the diseases that can be treated with the engineered immune cells or pharmaceutical compositions of the invention are selected from: leukemia, lymphoma, multiple myeloma, brain glioma, pancreatic cancer, gastric cancer, and the like.

In one embodiment, the method further comprises administering to the subject one or more additional chemotherapeutic agents, biological agents, drugs or treatments. In this embodiment, the chemotherapeutic agent, biological agents, drug or treatment is selected from radiation therapy, surgery, antibody agents and/or small molecules and any combination thereof.

The invention will be described in detail below with reference to the accompanying drawings and in conjunction with examples. It should be noted that those skilled in the art should understand that the accompanying drawings and the embodiments of the invention are only for the purpose of illustration, and do not constitute any limitation to the invention. The embodiments in the present application and the features in the embodiments may be combined with each other where there is no contradiction.

### DESCRIPTION OF DRAWINGS

FIG. 1 shows schematic design of a preferred embodiment of the invention.

FIG. 2 shows the expression level of CAR on Fite-CAR (18.2-18.2) T and Fite-CAR (19-22) T cells containing scFv.

FIG. 3 shows the killing effect of Fite-CAR (18.2-18.2) T cells on target cells.

FIG. 4 shows the secretion level of scFv-Fc in Fite-CAR (18.2-18.2) T and Fite-CAR (19-22) T cells.

FIG. 5 shows the secretion level of sdAb-Fc in Fite-CAR (18.2-18.2) X T cells.

FIG. 6 shows the killing effect of Fite-CAR (18.2-18.2) X T cells on target cells.

FIG. 7 shows the level of IFN-γ release from Fite-CAR (18.2-18.2) X T cells.

FIG. 8 shows the NK cell killing effect of Fite-CAR (18.2-18.2) X T cells.

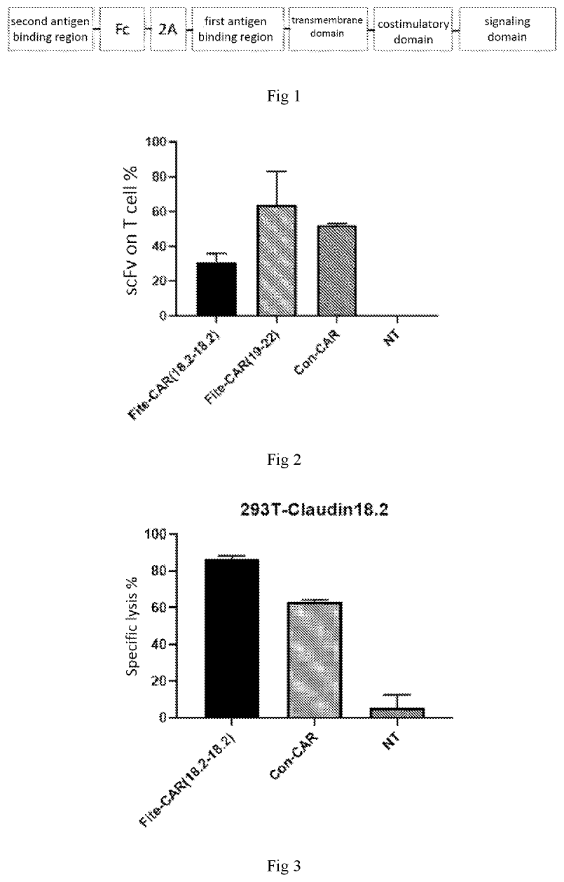

### EXAMPLES

The sequences used in the following examples are summarized in Table 1 below.

TABLE 1
Sequences used in the examples of the invention

| SEQ ID NO | Description |
|---|---|
| SEQ ID NO: 1 | Nucleotide sequence of anti-Claudin18.2 scFv1 |
| SEQ ID NO: 2 | Amino sequence of anti-Claudin18.2 scFv1 |
| SEQ ID NO: 3 | Nucleotide sequence of anti-CD19 scFv |
| SEQ ID NO: 4 | Amino Nucleotide sequence of anti-CD19 scFv |
| SEQ ID NO: 5 | Nucleotide sequence of anti-CD22 scFv |
| SEQ ID NO: 6 | Amino sequence of anti-CD19 scFv |
| SEQ ID NO: 7 | Nucleotide sequence of anti-Claudin18.2 sdAb |
| SEQ ID NO: 8 | Amino sequence of anti-Claudin18.2 sdAb |
| SEQ ID NO: 9 | Nucleotide sequence of Fc region |
| SEQ ID NO: 10 | Amino of Fc region |
| SEQ ID NO: 11 | Nucleotide sequence of CD8α transmembrane domain |
| SEQ ID NO: 12 | Amino sequence of CD8α transmembrane domain |
| SEQ ID NO: 13 | Nucleotide sequence of 4-1BB costimulatory domain |
| SEQ ID NO: 14 | Amino sequence of 4-1BB costimulatory domain |
| SEQ ID NO: 15 | Nucleotide sequence of CD3 ζ signaling domain |
| SEQ ID NO: 16 | Amino sequence of CD3 ζ signaling domain |
| SEQ ID NO: 17 | Nucleotide sequence of IgG linker |
| SEQ ID NO: 18 | Amino sequence of IgG linker |
| SEQ ID NO: 19 | Nucleotide sequence of CD8α signal peptide |
| SEQ ID NO: 20 | Amino sequence of CD8α signal peptide |
| SEQ ID NO: 21 | Nucleotide sequence of GM-CSFRα signal peptide |
| SEQ ID NO: 22 | Amino sequence of GM-CSFRα signal peptide |
| SEQ ID NO: 23 | Nucleotide sequence of F2A peptide |
| SEQ ID NO: 24 | Amino sequence of F2A peptide |
| SEQ ID NO: 25 | Nucleotide sequence of CD8α hinge region |
| SEQ ID NO: 26 | Amino sequence of CD8α hinge region |
| SEQ ID NO: 27 | Nucleotide sequence of anti-Claudin18.2 scFv2 |
| SEQ ID NO: 28 | Amino sequence of anti-Claudin18.2 scFv2 |

T cells used in all examples of the invention are primary human CD4+CD8+ T cells isolated from healthy donors using Ficoll Paque™ premium (GE Healthcare, cat. No. 17-5442-02) by leukapheresis.

### Example 1: Constructing Conventional CAR T Cells

Coding sequences of the following proteins were synthesized and cloned sequentially into a pGEM-T Easy vector (Promega, cat. No. a1360) to obtain a CAR plasmid: CD8α signal peptide, anti-Claudin18.2 scFv1, CD8α hinge region, CD8α transmembrane region, 4-1BB costimulatory domain, CD3 ζ Intracellular signaling domains. The correct insertion of the target sequence was confirmed by sequencing.

After diluting the above plasmid by adding 3 mL Opti-MEM (Gibco, cat. No. 31985-070) in sterile tubes, packaging vector psPAX2 (Addgene, cat. No. 12260) and envelope vector pMD2.G (Addgene, cat. No. 12259) were added according to the ratio of plasmid: viral packaging vector: viral envelope vector=4:2:1. Then, 120 μL X-treme GENE HP DNA transfection reagent (Roche, cat. No. 06366236001) was added, immediately mixed, incubated at room temperature for 15 min, and then plasmid/vector/transfection reagent mixture was added dropwise into the culture flask of 293T cells. Virus was collected at 24 h and 48 h, and after combining them, concentrated lentivirus was obtained by ultracentrifugation (25000 g, 4° C., 2.5 h).

T cells were activated with Dynabeads CD3/CD28 CTSTM (Gibco, cat. No. 40203d) and cultured at 37° C. and 5% CO2 for 1 day. Then, concentrated lentivirus was added, and after 3 days of continued culture, CAR T (ie, con-CAR T) cells targeting Claudin18.2 was obtained.

### Example 2: Constructing Fite-CAR T Cells

Construct Fite-CAR plasmids: the coding sequence of CD8α signal peptide, anti-Claudin18.2 scFv1, CD8α hinge region, CD8α transmembrane region, 4-1BB costimulatory domain, CD3 ζ intracellular signaling domain, F2A peptide, GM-CSFRα signal peptide, anti-Claudin18 2 scFv2, IgG linker peptide, Fc region was cloned into a pGEM-T Easy vector (Promega, cat. No. A1360) to obtain Fite-CAR (18.2-18.2) plasmid, and the correct insertion of the target sequence was confirmed by sequencing. The Fite-CAR (19-22) plasmid was obtained by the same method, in which scFv1 was anti-CD19 scFv1 (SEQ ID No: 3), scfv2 was anti-CD22 scFv (SEQ ID No: 5), and the remaining elements were the same as the Fite-CAR (18.2-18.2) plasmid.

After diluting the above plasmids with 3 mL Opti-MEM (Gibco, cat. No. 31985-070) in sterile tubes, packaging vector psPAX2 (addgene, cat. No. 12260) and envelope vector pMD2.G (addgene, cat. No. 12259) were added according to the ratio of plasmid: viral packaging vector: viral envelope vector=4:2:1. Then, 120 μL X-treme GENE HP DNA transfection reagent (Roche, cat. No. 06366236001) was added, immediately mixed, incubated at room temperature for 15 min, and then plasmid/vector/transfection reagent mixture was added dropwise into the culture flask of 293T cells. Viruses were collected at 24 h and 48 h, and after combining them, concentrated Fite-CAR (18.2-18.2) and Fite-CAR (19-22) lentiviruses were obtained respectively by ultracentrifugation (25000 g, 4° C., 2.5 h).

T cells were activated with Dynabeads CD3/CD28 CTSTM (Gibco, cat. No. 40203d) and cultured at 37° C. and 5% CO2 for 1 day. Then, concentrated Fite-CAR lentivirus was added, and after 3 days of continuous culture, Fite-CAR (18.2-18.2) T cells and Fite-CAR (19-22) T cells were obtained.

After 11 days of incubation at 37° C. and 5% CO2, the expression levels of scFv in Fite-CAR T cells were detected by flow cytometry, using Biotin-SP (long spacer) AffiniPure Goat Anti-Mouse IgG, F(ab′)₂ Fragment Specific (min X Hu, Bov, Hrs Sr Prot) (jackson immunoresearch, cat. No. 115-065-072) as primary antibody, APC Streptavidin (BD Pharmingen, cat. No. 554067) or PE Streptavidin (BD pharmingen, cat. No. 554061) as secondary antibody. The results are shown in FIG. 2 (NT is unmodified wild-type T cells).

As is shown in FIG. 2, both Fite-CAR (18.2-18.2) T cells and Fite-CAR (19-22) T cell cells express CAR efficiently.

### Example 3: Functional Validation of Fite-CAR T Cells

### 3.1 Detecting Killing Effects on Target Cells

When T cells kill target cells, the number of target cells will decrease. When T cells are co-cultured with target cells expressing luciferase, the secreted luciferase will decrease along with the decrease of the number of target cells. Luciferase catalyzes the conversion of fluorescein to oxidative fluorescein, during which oxidation bioluminescence occurs and the intensity of luminescence will depend on the level of luciferase expressed by the target cells. Therefore, the detected fluorescence intensity can reflect the killing ability of T cells on target cells.

The 293T-Claudin18.2 target cells used in this example are Claudin18.2 positive monoclonal cells selected by flow cytometry after infecting 293T cells with a lentivirus expressing Claudin18.2.

To test the killing effect of Fite-CAR (18.2-18.2) T cells against target cells, the 293T-Claudin18.2 target cells comprising the fluorescein gene were first spread into a 96-well plate at 1×10⁴/well, and then the Fite-CAR (18.2-18.2) T cells, Con-CAR T cells (positive control), and untransfected T cells (negative control) were spread into the 96-well plate for coculture at a effector cell: target cell ratio of 16:1, fluorescence was determined with a microplate reader after 16-18 hours. Killing efficiency was calculated according to the calculation formula: (Mean fluorescence of target cells−Mean fluorescence of sample)/Mean fluorescence of target cells×100%. Results are shown in FIG. 3.

As is shown in FIG. 3, compared with NT, Fite-CAR (18.2-18.2) T is more efficient in killing target cells, and the killing efficiency is much higher than that of Con-CAR T cells.

3.2 Detecting the Secretion Level of scFv-Fc

If Fite-CAR T cells can effectively secrete scFv-Fc region, it can be recognized by immune effector cells expressing Fc receptor (FcR), including NK cells, macrophages, and dendritic cells, thereby recruiting these immune effector cells to further enhance the killing effect on target cells. Therefore, the inventors used enzyme-linked immunosorbent assay (ELISA) to detect the secretion level of scFv-Fc in Fite-CAR T cells.

Fite-CAR (18.2-18.2) T, Fite-CAR (19-22) T, Con-CAR T and NT cells were respectively incubated in x-vivo 15 medium (Lonza, cat. No. 04-418Q) without IL-2 at 37° C., 5% CO2. After 24 h, the culture was collected and centrifuged at 1600 rpm for 5 min at 4° C. to obtain a cell culture supernatant.

96-well plates were coated with capture antibody Recombinant Human Claudin-18.2 (N-8His) (Novoprotein, Cat. No. CR53) or CD22 Protein, Human, Recombinant (His Tag) (sino biological, Cat. No. 11958-H08H), and incubated overnight at 4° C., then the supernatant was removed, 250 μL of PBST (1×PBS with 0.1% Tween) solution containing 2% BSA (sigma, Cat. No. V900933-1 kg) was added, and incubated at 37° C. for 2 hours. After removing the supernatant, 250 μL of PBST (1×PBS containing 0.1% Tween) was added and washed 3 times. 50 μL of cell culture supernatant was then added to each well and incubated for 1 hour at 37° C. The supernatant was removed, then 250 μL of PBST (1×PBS with 0.1% Tween) was added and washed 3 times. Then 50 μL of detection antibody HRP Goat anti-mouse IgG (Biolegend, Cat. No. 405306) was added to each well and incubated at 37° C. for 30 minutes (or, in the case of detection of CD22 scFv-Fc, detection antibody Biotin-SP (long spacer) AffiniPure Goat Anti-Human IgG, F(ab′)₂ fragment specific (Jackson immunoresearch, Cat. No. 109-065-097) is used, washed with 250 μL PBST (1×PBS with 0.1% Tween) after 1 hour incubation at 37° C. 3 times. HRP Streptavidin (Biolegend, Cat. No. 405210) was added and incubated at 37° C. for 30 minutes). The supernatant was discarded, 250 μL PBST (1×PBS containing 0.1% Tween) was added, and washed 5 times.

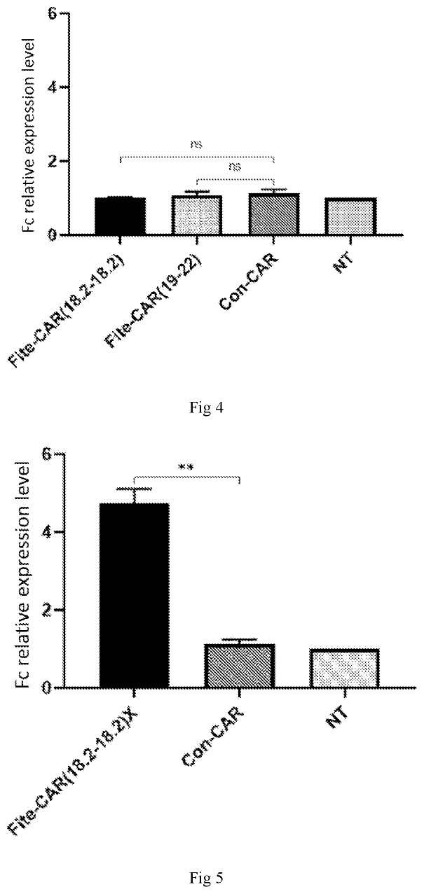

50 μL of TMB substrate solution was added to each well. The reaction carried out in the dark at room temperature for 30 minutes, after which 50 μL, of 1 mol/L H₂SO₄ was added to each well to stop the reaction. Within 30 minutes after stopping the reaction, the absorbance at 450 nm was detected using a microplate reader, and the relative expression level of the scFv-Fc fusion polypeptide in the supernatant was calculated by the ratio to the read value of the NT cell culture supernatant, and the results are shown in FIG. 4.

Surprisingly, no significant expression of scFv-Fc was detected in either Fite-CAR T cell supernatant compared to Con-CAR T and NT cells. This might due to the fact that the antigen-binding regions in the two Fite-CAR T were both scFv structures, which caused the adhesion of the VL and VH domains in the two scFv structures with each other, thus affecting the normal secretion of scFv-Fc.

In summary, since the two Fite-CAR T cells failed to effectively secrete scFv-Fc, they could not recruit other immune cells to enhance the killing effect of CAR T cells on target cells.

### Example 4: Constructing Fite-CARX T Cells

Due to the unique VHH structure of a single domain antibody (sdAb), i.e., it contains only heavy chain regions, makes it promising to overcome the problem that scFv-Fc cannot be secreted because of the potential VL adhesions with VH domains, which is found in Example 3. Thus, the inventors replaced one of the scFv structures with a single domain antibody (sdAb) to obtain Fite-CARX T cells.

Specifically, the coding sequences of the CD8α signal peptide, anti-Claudin18.2 scFv1, CD8α hinge region, CD8α transmembrane region, 4-1BB costimulatory domain, CD3ζ signaling domain, F2A peptide, GM-CSFRα signal peptide, anti-Claudin18.2 sdAb, IgG linker peptide, Fc region, were cloned into a pGEM-T Easy vector (Promega, Cat. No. A1360) in the order of CD8α signal peptide-sdAb-linker peptide-Fc region-2A peptide-GM-CSFRα signal peptide-scFv1-hinge region-transmembrane region-costimulatory domain-signaling domain from 5′ to 3′, to obtain a Fite-CAR (18.2-18.2)X plasmid, and the correct insertion of the target sequence was confirmed by sequencing.

According to the procedure described in Example 2, the plasmid was packaged with lentivirus using 293T cells, and T cells were infected to obtain Fite-CAR (18.2-18.2)X T cells.

### Example 5: Functional Validation of Fite-CAR (18.2-18.2)X T Cells

The secretion level of the sdAb-Fc fusion polypeptide of the Fite-CAR (18.2-18.2)X T cells was detected by ELISA using capture antibody Recombinant Human Claudin-18.2 (N-8His) (Novoprotein, Cat. No. CR53) according to the method of Example 3.2, and the results are shown in FIG. 5.

As shown in FIG. 5, compared with Con-CAR T cells and NT cells, more significantly secreted sdAb-Fc fusion polypeptide was detected in the supernatant of Fite-CAR (18.2-18.2)X T, indicating that the single-domain antibody structure can effectively avoid the mutual adhesion between scFv, and thus promote the secretory expression of Fc fusion polypeptide.

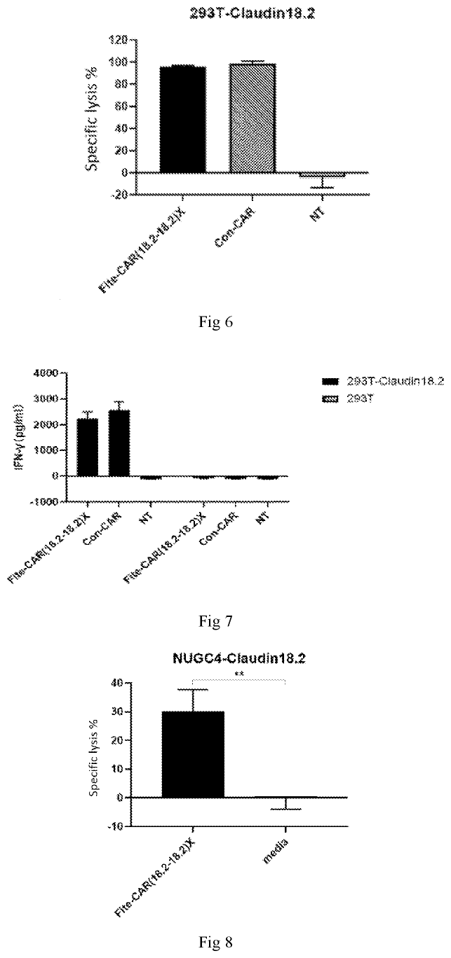

In addition, the killing effect of Fite-CAR (18.2-18.2)X T cells on the 293T-Claudin18.2 target cells was detected according to the method described in example 3.1, and the results are shown in FIG. 6.

As shown in FIG. 6, compared with NT, T cells comprising Fite-CAR (18.2-18.2)X were able to kill target cells more effectively, and the killing efficiency was comparable to that of Con-CAR T cells.

### Example 6: Cytokine Release by Fite-CAR (18.2-18.2)X T Cells

When T cells kill target cells, the number of target cells decreases and cytokines such as IL2 and IFN-γ are released. Enzyme-linked immunosorbent assay (ELISA) was used to detect the release level of IFN γ when Fite-CAR (18.2-18.2)X T cells killed target cells according to the following steps.

### (1) Collecting Cell Coculture Supernatant

Target cells 293T-Claudin18.2 and non-target cells 293T were respectively spread in 96-well plates at 1×10⁵/well. Then Fite-CAR (18.2-18.2) X T, Con-CAR T (positive control) and NT cells (negative control) were co-cultured with target cells and non-target cells respectively at a 1:1 ratio. After 18-24 hours, cell co-culture supernatant was collected.

### (2) Detecting the Secretion Level of IFN γ in the Supernatant

The 96-well plate was coated with capture antibody Purified anti-human IFN-γ Antibody (Biolegend, Cat. No. 506502) and incubated overnight at 4° C., then the antibody solution was removed and 250 μL PBST (0.1% Tween in 1×PBS) solution containing 2% BSA (sigma, Cat. No. V900933-1 kg) was added, incubated at 37° C. for 2 hours. Plates were then washed 3 times with 250 μL PBST (1×PBS containing 0.1% Tween). 50 μL of cell co-culture supernatant or standards was added to each well and incubated at 37° C. for 1 hour, then washed 3 times with 250 μL of PBST (1×PBS containing 0.1% Tween). Then, 50 μL of detection antibody Anti-Interferon gamma antibody [MD-1] (Biotin) (abcam, Cat. No. ab25017) was added to each well, incubated at 37° C. for 1 hour, and washed with 250 μL of PBST (1×PBS containing 0.1% Tween) plate 3 times. Then HRP Streptavidin (Biolegend, Cat. No. 405210) was added, incubated at 37° C. for 30 minutes, the supernatant was discarded, 250 μL PBST (1×PBS containing 0.1% Tween) was added, and washed 5 times. 50 μL of TMB substrate solution was added to each well. The reaction was carried out at room temperature for 30 minutes in the dark, after which 50 μL of 1 mol/L H₂SO₄ was added to each well to stop the reaction. Within 30 minutes after stopping the reaction, a microplate reader was used to detect the absorbance at 450 nm, and the content of cytokines was calculated according to the standard curve (drawn according to the reading and concentration of the standard). The results are shown in FIG. 7.

As shown in FIG. 7, the release of IFN γ was not detected in non-target cells 293T, but was detected in target cell 293T-Claudin18.2, indicating that the killing effects of both Con-CART cells and Fite-CAR (18.2-18.2) X T cells were specific. Furthermore, the release level of IFN-γ of Fite-CAR (18.2-18.2)X T cells is comparable to Con-CAR T cells when killing target cells.

### Example 7: The Killing Effect of NK Cells on Target Cells Mediated by Fite-CAR (18.2-18.2)X T Cells

Since Fite-CAR (18.2-18.2)X T cells can significantly secrete sdAb-Fc fusion polypeptide, the inventors further tested whether it is capable of mediating NK cells for tumor killing.

The NK cells used in this example were obtained by grinding the mouse spleen, adding a mouse spleen lymphocyte separation solution (TBD, Cat. No. LTS1092PK-200), and centrifuging to obtain white membrane cells. Then, PE anti-mouse NK1.1 (Biolegend, Cat. No. 108701) and Anti-PE Microbeads (MiltenyiArt, Cat. No. 130-048-801) were added and subjected to positive screening on a magnetic rack to obtain NK1.1 positive cells.

The NUGC4-Claudin18.2 target cell used in this example is a Claudin18.2 positive monoclonal cell selected by flow cytometry after infection of NUGC4 cells with a lentivirus expressing Claudin18.2 antigen and luciferase.

NUGC4-Claudin18.2 comprising the fluorescein gene were spread into 96-well plates at 1×10⁴/well. Then, the NK cells were re-suspended using Fite-CAR (18.2-18.2)X T cell supernatant and fresh medium (media), respectively, and the re-suspended NK cells were added into a 96-well plate for co-culture at an effector-to-target ratio of 4:1 (i.e., the ratio of effector NK cells to target cells). After 16-18 h, the fluorescence values were measured by a microplate reader. According to the calculation formula: (Mean fluorescence of target cells—Mean fluorescence of sample)/Mean fluorescence of target cells×100%, the killing efficiency was calculated, and the results are shown in FIG. 8.

As shown in FIG. 8, compared with NT, Fite-CAR (18.2-18.2)X T cell supernatant can effectively mediate the killing effect of NK cells on NUGC4-Claudin18.2 target cells, and the effect was significantly higher than that in the fresh medium control group.

It should be noted that the above are only preferred embodiments of the invention, and are not intended to limit the invention, and those skilled in the art know that the invention may have various modifications and changes. It is understood by those skilled in the art that any modification, equivalent replacement, improvement made within the spirit and principle of the invention shall be included within the protection scope of the invention.

1. An engineered immune cell comprising:
(a) a first nucleic acid sequence encoding a chimeric antigen receptor or a chimeric antigen receptor encoded thereby, wherein the chimeric antigen receptor comprises a first antigen binding region, a transmembrane domain, and an intracellular signaling domain; and
(b) a second nucleic acid sequence encoding an Fc fusion polypeptide or an Fc fusion polypeptide encoded thereby, wherein the Fc fusion polypeptide comprises a second antigen binding region and an Fc region,
wherein the first antigen binding region and the second antigen binding region are not scFv at the same time.

2. The immune cell of claim 1, wherein the first antigen binding region and the second antigen binding region bind the same antigen.

3. The immune cell of claim 1, wherein the first antigen binding region and the second antigen binding region bind different antigens.

4. The immune cell of claim 1, wherein the first antigen binding region and the second antigen binding region are selected from scFv, sdAb, nanobodies, antigen binding ligands, recombinant fibronectin structures Domain, and DARPIN.

5. The immune cell of claim 4, wherein the first antigen binding region is an scFv and the second antigen binding region is an sdAb or nanobody, or the first antigen binding region is an sdAb or nanobody and the second antigen binding region is an scFv.

6. The immune cell of claim 1, wherein the first antigen binding region and the second antigen binding region are selected from monoclonal antibodies, polyclonal antibodies, recombinant antibodies, human antibodies, humanized antibodies, murine antibodies and chimeric antibodies.

7. The immune cell of claim 1, wherein the first antigen binding region and the second antigen binding region bind to a target selected from TSHR, CD19, CD123, CD22, BAFF-R, CD30, CD171, CS-1, CLL-1, CD33, EGFRvIII, GD2, GD3, BCMA, GPRC5D, Tn Ag, PSMA, ROR1, FLT3, FAP, TAG72, CD38, CD44v6, CEA, EPCAM, B7H3, KIT, IL-13Ra2, mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Claudin18.2, Prostase, PAP, ELF2M, Ephrin B2, IGF-I receptor, CAIX, LMP2, gploo, bcr-abl, tyrosinase, EphA2, Fucosyl GM1, sLe, GM3, TGS5, HMWMAA, o-acetyl Base-GD2, Folate receptor beta, TEM1/CD248, TEM7R, CLDN6, GPRC5D, CXORF61, CD97, CD 179a, ALK, polysialic acid, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, Podin, HPV E6, E7, MAGE A1, ETV6-AML, sperm protein 17, XAGE1, Tie 2, MAD-CT-1, MAD-CT-2, Fos-associated antigen 1, p53, p53 mutants, prostate specific protein, survivin and telomerase, PCTA-1/Galectin 8, MelanA/MART1, Ras mutants, hTERT, sarcoma translocation breakpoint, ML-IAP, ERG (TMPRSS2 ETS fusion gene), NA17, PAX3, androgen receptor, Cyclin B1, MYCN, RhoC, TRP-2, CYP1B 1, BORIS, SART3, PAX5, OY-TES 1, LCK, AKAP-4, SSX2, RAGE-1, human telomerase reverse transcriptase, RU1, RU2, intestinal carboxylesterase, mut hsp70-2, CD79a, CD79b, CD72, LAIR1, FCAR, LILRA2, CD300LF, CLEC12A, BST2, EMR2, LY75, GPC3, FCRL5, IGLL1, PD1, PDL1, PDL2, TGF β, APRIL, NKG2D, and any combination thereof.

8. The immune cell of claim 7, wherein the target is selected from CD19, CD20, CD22, BAFF-R, CD33, EGFRvIII, BCMA, GPRC5D, PSMA, ROR1, FAP, ERBB2 (Her2/neu), MUC1, EGFR, CAIX, WT1, NY-ESO-1, CD79a, CD79b, GPC3, Claudin18.2, NKG2D, and any combination thereof.

9. The immune cell of claim 1, wherein the transmembrane domain is selected from the transmembrane domains of the following proteins: TCR α chain, TCR β chain, TCR γ chain, TCR δ chain, CD3 ζ subunit, CD3 ε subunit, CD3 γ subunit, CD3 δ subunit, CD45, CD4, CD5, CD8 α, CD9, CD16, CD22, CD33, CD28, CD37, CD64, CD80, CD86, CD134, CD137 and CD154.

10. The immune cell of claim 1, wherein the intracellular signaling domain is selected from the signaling domains of the following proteins: FcR γ, FcR β, CD3 γ, CD3 δ, CD3 ε, CD3 ζ, CD22, CD79a, CD79b and CD66d.

11. The immune cell of claim 1, wherein the chimeric antigen receptor further comprises one or more costimulatory domains.

12. The immune cell of claim 11, wherein the costimulatory domain is a costimulatory signaling domain selected from TLR1, TLR2, TLR3, TLR4, TLR5, TLR6, TLR7, TLR8, TLR9, TLR10, CARD11, CD2, CD7, CD8 α, CD18 (LFA-1), CD27, CD28, CD30, CD40, CD54 (ICAM), CD83, CD134 (OX40), CD137 (4-1BB), CD270 (HVEM), CD272 (BTLA), CD276 (B7-H3), CD278 (ICOS), CD357 (GITR), DAP10, LAT, NKG2C, SLP76, PD-1, LIGHT, TRIM and ZAP70.

13. The immune cell of claim 1, wherein the Fc region comprises a CH2 domain and a CH3 domain.

14. The immune cell of claim 1, wherein the first nucleic acid sequence and the second nucleic acid sequence are located in different vectors.

15. The immune cell of claim 1, wherein the first nucleic acid sequence and the second nucleic acid sequence are located in the same vector.

16. The immune cell of claim 14, wherein the vector is a linear nucleic acid molecule, plasmid, retrovirus, lentivirus, adenovirus, vaccinia virus, Rous sarcoma virus (RSV), polyoma virus and Adeno-associated virus (AAV), phage, cosmid or artificial chromosome.

17. The immune cell of claim 1, wherein said immune cell is selected from T cells, macrophages, dendritic cells, monocytes, NK cells or NKT cells.

18. The immune cell of claim 17, wherein the immune cell is a T cell selected from CD4+/CD8+ double positive T cells, CD4+ helper T cells, CD8+ T cells, tumor infiltrating cells, memory T cells, naive T cells, γ δ-T cells and α β-T cells.

19. A pharmaceutical composition comprising the immune cell of claim 1 and one or more pharmaceutically acceptable excipients.

20-21. (canceled)

22. A kit comprising one or more vectors, wherein the vector comprises:
(a) a first nucleic acid sequence encoding a chimeric antigen receptor, wherein the chimeric antigen receptor comprises a first antigen binding region, a transmembrane domain, and an intracellular signaling domain; and
(b) a second nucleic acid sequence encoding an Fc fusion polypeptide, wherein the Fc fusion polypeptide comprises a second antigen binding region and an Fc region,
wherein the first antigen binding region and the second antigen binding region are not scFv at the same time.

23-24. (canceled)
